(19) United States
(12) Patent Application Publication
(10) Pub. No.: US 2008/0099025 A1
(43) Pub. Date: May 1, 2008

(54) Tracheal tube apparatus

(75) Inventor: Nicholas Paul MacMillan, Dover (GB)

Correspondence Address: LOUIS WOO;LAW OFFICE OF LOUIS WOO, 717 NORTH FAYETTE STREET, ALEXANDRIA, VA, 22314, US

(73) Assignee: Smiths Group plc, London (GB)

(21) Appl. No.: 11/976,971

(22) Filed: Oct. 30, 2007

(30) Foreign Application Priority Data
Oct. 31, 2006 (GB) 0621608.9

(51) Int. Cl. A61M 16/04

(52) U.S. Cl. 128/207.15

(57) ABSTRACT

An arrangement for automatic suctioning above the cuff of a tracheal tube has a suction lumen extending along the tube and opening at one end at an opening on the outside of the tube just above the cuff. A suction line connects with the other end of the lumen and extends to a source of suction. A timer is connected in the line, between the tube and the suction source. The timer may be clockwork and is arranged to prevent suctioning flow along the suction line except for short periods, so that secretions that collect above the cuff are suctioned periodically without the need for intervention by a nurse or clinician.

### BACKGROUND OF THE INVENTION

This invention relates to tracheal tube apparatus.

Where a patient is ventilated by a tracheal tube, such as an endotracheal or tracheostomy tube, an inflated cuff on the tube is used to seal the outside of the tube with the trachea so that gas is confined to flow along the bore of the tube. One problem this causes is that secretions from the upper part of the respiratory system collect in the trachea outside the tube above the cuff. It is important periodically to remove these secretions in order to reduce the accumulation of bacteria, which is thought can give rise to ventilator-associated pneumonia (VAP). One way in which these secretions can be removed is by inserting a small-bore suction tube down the trachea outside the tube so that its tip is located close to the upper end of the inflated cuff. This procedure can be quite difficult and time consuming, it also requires special procedures to dispose of the used suction tube in order to prevent cross-contamination. An alternative way in which secretions can be removed is by use of a tracheal tube incorporating a built-in suction lumen, such as of the kind described in U.S. Pat. No. 4,674,495, U.S. Pat. No. 5,143,062, U.S. Pat. No. 6,062,223, U.S. Pat. No. 6,460,540, U.S. Pat. No. 2,854,982, U.S. Pat. No. 4,305,392, U.S. Pat. No. 4,762,125, U.S. Pat. No. 5,067,497, U.S. Pat. No. 5,201,310, U.S. Pat. No. 5,311,864, U.S. Pat. No. 5,372,131, U.S. Pat. No. 5,501,215, U.S. Pat. No. 5,582,167, U.S. Pat. No. 5,819,723, EP1219317, U.S. Pat. No. 4,840,173, WO99/38548, JP290023/1997, JP5340/1998, JP337326/1997 U.S. Pat. No. 4,632,108, U.S. Pat. No. 5,143,062, DE19533615, DE4445428, DE3523663, WO99/07428 and WO96/37250. U.S. Pat. No. 4,607,635 describes an alternative arrangement having a channel open at various locations along its length and through which a suction catheter can be inserted. There are also situations where it can be desirable to remove secretions that collect on the inside of a tracheal tube, as described in U.S. Pat. No. 5,832,920. A problem with these arrangements is that they all rely on intervention by hospital staff to connect the suction lumen periodically to a suction source. In a busy hospital environment this procedure may be missed or not carried out as frequently as is desirable. Similar problems exist where it is necessary periodically to supply fluid, such as irrigating fluid or medication, to the tube.

### BRIEF SUMMARY OF THE INVENTION

It is an object of the present invention to provide alternative tracheal tube apparatus.

According to one aspect of the present invention there is provided apparatus including a tracheal tube, a fluid passage opening on the tube by which fluid can be supplied to or from the inside or outside the tube, a source, and a connection between the fluid passage and the source, the apparatus including timer means for automatically enabling or preventing flow along the fluid passage to or from the source.

The timer means is preferably connected in line between the fluid passage and the source. The timer means may be spring driven, having a knob that is rotatable to tension a spring. Alternatively, the timer means may be electrically driven. The tracheal tube preferably includes a sealing cuff on its outside close to its patient end, the fluid passage opening being on the outside of the tube adjacent the upper end of the cuff. The source may be a source of suction or a source of irrigating fluid or medication. The timer means may include a valve. Alternatively, the timer means may be arranged to occlude a passage through tubing when flow is prevented.

Apparatus according to the present invention will now be described, by way of example, with reference to the accompanying drawings.

### BRIEF DESCRIPTION OF THE DRAWINGS

FIG. 1 shows the apparatus in a simplified form;

FIG. 2 is a perspective view of a first form of timer means;

FIG. 3 is a perspective view of a second form of timer means; and

FIG. 4 is a perspective view of a third form of timer means.

### DETAILED DESCRIPTION OF THE PREFERRED EMBODIMENTS

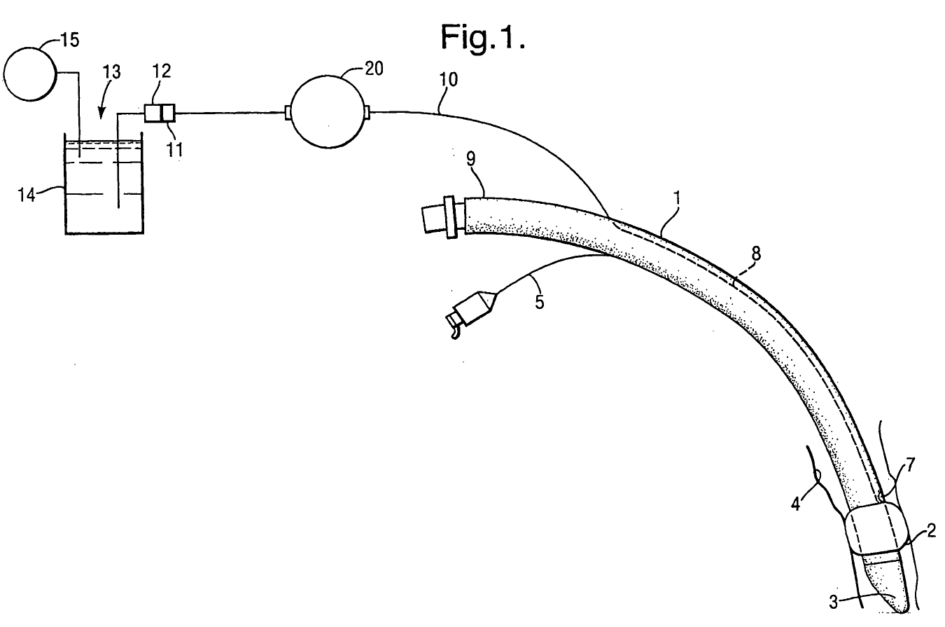

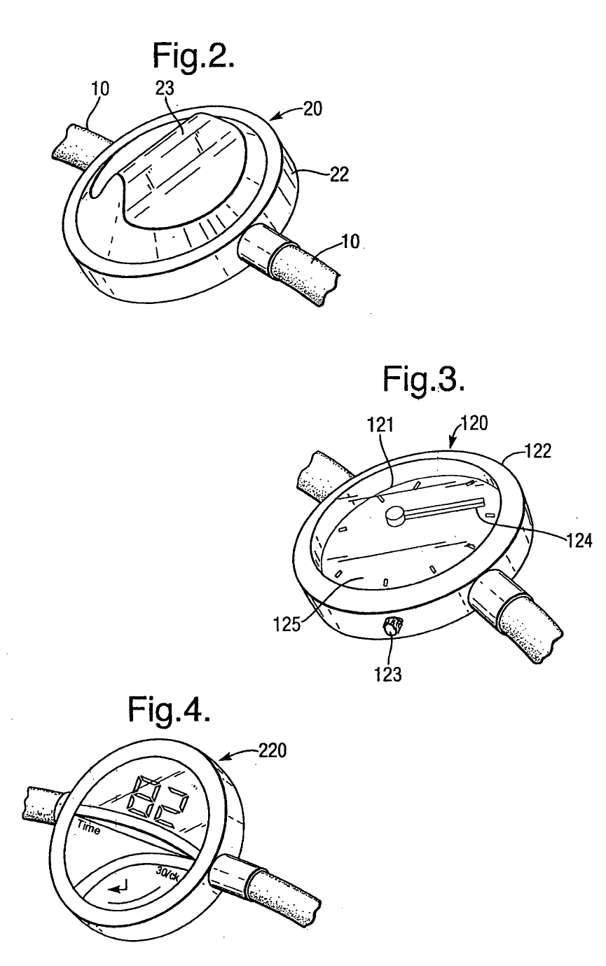

With reference first to FIGS. 1 and 2, the apparatus includes an endotracheal tube 1 having a cuff 2 close to its patient end 3 that seals with the trachea 4 so that gas is confined to flow along the bore of the tube. The cuff 2 is inflated and deflated via an inflation line 5, in the usual way. The tube 1 also includes a fluid or suction aperture 7 located close to the upper end of the cuff 2 in the manner described in U.S. Pat. No. 5,201,310 and opening on the external surface of the tube. The aperture 7 communicates with a fluid or suction lumen 8 extruded within the wall of the tube 1 and connected towards the rear, machine end 9 of the tube with a small-bore fluid or suction line 10. The line 10 differs from conventional suction lines in that it incorporates timer means 20 at a location between its ends by which flow along the line is controlled, in a manner described later.

The rear end of the suction line 10 is terminated by a coupling 11 connected to the inlet 12 of a suction source 13. Typically, the suction source 13 is provided by a suction container 14 connected to a wall-mounted suction outlet 15.

The timer means 20 may take various different forms each of which is arranged to maintain the suction line 10 normally closed so that no suction is applied at the suction aperture 7 but automatically enables flow through the suction line 10 for a predetermined, shorter time period and then closes the suction line again. In one form the timer means 20 could simply perform one cycle so, once set by the user, it maintains suction off for a set period and then enables one suction period before turning off again. With such a timer, the nurse would set the timer to ensure that the next suction period was not missed. Alternatively, the timer means 20 could be arranged automatically repeatedly to turn suctioning on and off for several cycles.

In the form shown in FIG. 2, the timer means 20 is a spring-driven, clockwork device having a circular housing 22 with a rotatable knob 23 on one face. Inside the housing 22 the knob 23 is connected with a spring and a conventional mechanical regulator mechanism (neither shown) so that the knob can be twisted manually in a clockwise direction to tension the spring and, when released, rotates in the opposite direction, anticlockwise at a slower, regulated rate. Inside the housing 22 the knob 23 is coupled to some means for controlling flow of fluid along the suction line. This could take many different forms. For example, it could be in the form of a valve connected in line with the suction line 10 and coupled mechanically with the knob 23 so that the valve is maintained closed for most of the time but is periodically opened, such as every several hours for a period of about a minute.

Alternatively, the housing could include a short length of a resilient tubing connected in line with the suction line, The knob could be formed with cam surfaces inside the housing arranged to bear on the resilient tubing and squeeze it and occlude the passage through the tubing except for short periods when suctioning is required.

FIG. 3 illustrates an alternative timer 120 having a housing 122 with a transparent face 121 and a rotatable hand 124 movable around a circular, calibrated dial 125. Preferably the calibrations of the dial 125 indicate periods when the timer allows flow along the suction line 10. The timer 120 has a winder 123 on one side by which a clockwork mechanism in the timer can be wound up.

FIG. 4 illustrates an electrically-driven timer 220 where power is provided by a battery, a photo-voltaic cell, a fuel cell or the like (not shown). This timer 220 includes an electrically-driven valve or a motor-driven cam bearing on a resilient tube.

If the timer can be made at low cost it may be disposed of with the endotracheal tube 1. Alternatively, the timer may have a separate length of tubing with connectors so that it can be connected between the free end of the suction line of the endotracheal tube and the inlet of the suction container. In this way, the timer and its connecting tubing could be retained for use with several tubes.

In an alternative arrangement, the timer may have a path, such as a slot, into which a separate tube can be inserted, the timer having a mechanism for bearing on the inserted tube to occlude its passage except when suctioning is required. After use, the tube would be removed from the timer and disposed of but the timer would be used again repeatedly.

Apparatus of the present invention can be used to control supply of fluid to the tracheal tube, such as irrigation fluid or medication, as well as suctioning of fluid from the vicinity of the tube.

It will be appreciated that there are many other timer mechanisms that could be used automatically to occlude and open a path between a fluid source and a fluid aperture on a tracheal tube.

What I claim is:

1. Apparatus comprising: a tracheal tube having an outside and an inside, a fluid passage opening on the tube by which fluid can be supplied to or from the inside or outside the tube, a source, a connection between said fluid passage and said source, wherein the apparatus includes a timer arranged automatically to enable or prevent flow along the fluid passage to or from said source.

2. Apparatus according to claim 1, wherein said timer is connected in line between said fluid passage and said source.

3. Apparatus according to claim 1, wherein said timer is spring driven.

4. Apparatus according to claim 3, wherein said timer has a knob that is rotatable to tension a spring.

5. Apparatus according to claim 1, wherein said timer is electrically driven.

6. Apparatus according to claim 1, wherein the tracheal tube includes a sealing cuff on its outside close to its patient end, and wherein said fluid passage opening is on the outside of the tube adjacent the upper end of said cuff.

7. Apparatus according to claim 1, wherein said source is a source of suction.

8. Apparatus according to claim 1, wherein said source is a source of irrigating fluid or medication.

9. Apparatus according to claim 1, wherein said timer includes a valve.

10. Apparatus according to claim 1, wherein said timer is arranged to occlude a passage through tubing when flow is prevented.

11. Apparatus comprising: a tracheal tube having an outside, an inside, a patient end and a machine end; an inflatable cuff extending around the outside of the tube close to its patient end; a fluid passage opening on the outside of the tube adjacent said cuff and on its machine side; a source of suction; and a fluid suction line extending between said fluid passage and said suction source, wherein the apparatus includes a timer connected in said fluid suction line and arranged automatically to enable or prevent suction being applied where said fluid passage opens adjacent the cuff.

12. Apparatus according to claim 11, wherein said timer is driven by a spring.

13. Apparatus according to claim 11, wherein said timer is electrically driven.

14. Apparatus according to claim 11, wherein the fluid passage includes a lumen extending within a wall of the tube, wherein the lumen opens at an opening at one end on the outside of the tube, and wherein said suction line is a small-bore tube connected at an opposite end of said lumen.
